(19) United States
(12) Patent Application Publication
(10) Pub. No.: US 2012/0010485 A1
(43) Pub. Date: Jan. 12, 2012

(54) Device for Detecting at Least One Vital Parameter of a Person in a Vehicle and Device for Monitoring at Least One Vital Parameter of a Person in a Vehicle

(76) Inventors: Robert Couronne, Erlangen (DE); Hans-Joachim Moersdorf, Fuerth (DE); Andreas Tobola, Hemhofen (DE); Sergey Ershov, Erlangen (DE); Hans-Josef Gassmann, Meerbeck (DE); Rainer Haevescher, Stemwede (DE); Ludger Rake, Diepholz (DE); Joerg Meyer, Wagenfeld (DE)

(21) Appl. No.: 13/081,206

(22) Filed: Apr. 6, 2011

Related U.S. Application Data
(63) Continuation of application No. PCT/EP2009/006876, filed on Sep. 23, 2009.

(30) Foreign Application Priority Data
Oct. 7, 2008 (DE) 102008050639.7
Nov. 6, 2008 (DE) 102008056250.5

(51) Int. Cl. A61B 5/1455; A61B 6/00

(52) U.S. Cl. 600/340; 600/407

(57) ABSTRACT

A device for detecting at least one vital parameter of a person in a vehicle is described, having: an optoelectronic sensor arrangement for detecting the at least one vital parameter by means of light remission, wherein the optoelectronic sensor arrangement has at least one light source and a light-sensitive element, and wherein the first light source and the light-sensitive element are arranged in a finger bed of an operating element of the vehicle, and wherein the finger bed is implemented to integrate a fingertip of the person flush in a sensor area of the finger bed in which the first light source and the light sensitive element are arranged.

### CROSS-REFERENCE TO RELATED APPLICATIONS

This application is a continuation of copending International Application No. PCT/EP2009/006876, filed Sep. 23, 2009, which is incorporated herein by reference in its entirety, and additionally claims priority from German Applications Nos. DE 102008050639.7, filed Oct. 7, 2008 and DE 102008056250.5-35, filed Nov. 6, 2008, which are all incorporated herein by reference in their entirety.

### BACKGROUND OF THE INVENTION

The present application relates to a device for detecting and/or monitoring at least one vital parameter of a person, e.g. in a vehicle.

Known methods for detecting vital parameters are, for example, the optical plethysmography and pulse oxymetry. Here, the optical plethysmography and pulse oxymetry basically are based on the same measurement methods and represent a method for a non-invasive determination of the pulse rate, pulse rate variability and arterial oxygen saturation via the measurement of light absorption or light remission in the tissue. Here, the optical plethysmography is based on the changes of light absorption conditioned by volume variations of body fluids in vessels, e.g. blood, while pulse oxymetry is based on the different light absorption or light remission of a red and an infrared light-beam in screening skin and tissue.

The optical plethysmography and the pulse oxymetry have become part of clinical settings and are used both for standard monitoring of patients and also for diagnostic purposes.

Among others, pulse oxymetry is used more and more for “home care”, i.e. for “monitoring” the patient in his home environment. The main applications here are: a) taking care of patients with cardiologic risk factors, b) diagnostics of sleep disturbances, 3) detection of fatigue, d) detection of stress and e) chronic lung diseases. In pulse oxymetry, as a standard, the SpO₂ values are taken via an optical sensor at the finger, toe or earlobe. The measurement is typically executed using a clip sensor or an adhesive sensor. To be able to execute the pulse oxymetric measurements not only in the hospital, the patient typically receives a mobile device whose application part or sensor is directly attached to the body. This restricts mobility of the patient and is not acceptable as a solution in a vehicle mainly due to the restriction of mobility.

It was partially proposed to integrate optical sensors for the above-mentioned measurement methods into the steering-wheel rim. Here, however, frequently, the bad signal quality of optical measurement resulted to be a problem.

### SUMMARY

According to an embodiment, a device for detecting at least one vital parameter of a person in a vehicle, may have an optoelectronic sensor arrangement for detecting the at least one vital parameter by means of light remission, wherein the optoelectronic sensor arrangement has at least one light source and a light-sensitive element, wherein the first light source and the light-sensitive element are arranged in a finger bed of an operating element of the vehicle, wherein the finger bed is implemented to integrate a fingertip of the person flush in a sensor area of the finger bed in which the first light source and the light sensitive element are arranged, and wherein the finger bed is a recess open to one side in the operating element, which is implemented to receive a bottom side of the fingertip flush.

According to another embodiment, a device for monitoring at least one vital parameter of a person in a vehicle may have a device for detecting the at least one vital parameter of a person in a vehicle, which may have an optoelectronic sensor arrangement for detecting the at least one vital parameter by means of light remission, wherein the optoelectronic sensor arrangement has at least one light source and a light-sensitive element, wherein the first light source and the light-sensitive element are arranged in a finger bed of an operating element of the vehicle, wherein the finger bed is implemented to integrate a fingertip of the person flush in a sensor area of the finger bed in which the first light source and the light sensitive element are arranged, and wherein the finger bed is a recess open to one side in the operating element, which is implemented to receive a bottom side of the fingertip flush; and a device for evaluating the at least one vital parameter which is implemented to compare a value of the at least one vital parameter to at least one given threshold value and to output a warning message when the value of the at least one vital parameter exceeds or falls below this at least one threshold value.

Embodiments of the invention are based on the finding that complicated and extreme lighting conditions in the vehicle, in particular at the steering-wheel rim, form extraneous light artifacts which may distort the measurement.

The invention is based on the finding that prior solutions for the integration of optical sensors into automobile or vehicle environments, e.g. the integration of an optical sensor into the steering-wheel, are difficult or not at all to be realized in the automobile conditions. The reasons herefore are in particular non-suitable integration locations and the bad signal quality of optical measurement which will be referred to in detail in the following.

The steering-wheel rim has too little room for the integration of the optical sensors: Here, the finger bed or finger board may only be implemented restrictedly. A small area of the contacting location “finger sensor” leads to a non-optimum distribution of the contact pressure and thus to the corruption or even the loss of measurement data. In contrast to this, e.g. the gear lever knob offers sufficient space to implement the finger bed and thus enables a reliable detection of the measurement data.

Apart from this, the steering-wheel is one of the most flexible operating elements of the car or one of the operating elements which is moved most. Thus, the optical measurements will contain a large portion of movement artifacts, which again affect the detection of the measurement data or vital parameters.

Apart from this, great temperature fluctuations at the steering-wheel rim per se affect the measurement values strongly.

Embodiments of the device for the optical detection of at least one vital parameter comprise a light-sensitive element, e.g. an optical sensor, which is firmly integrated into an operating element of the automobile or vehicle. No further equipment is needed which the driver wears on his body, so that the impairment of the driver is kept as low as possible.

Operating elements of the vehicle are, for example, the gear lever knob for the steering-wheel, and in the broader sense also include other elements of the automobile which the driver, but also a passenger contacts, like, for example, the middle or side arm rests.

Embodiments of the device for detecting at least one vital parameter enable, thanks to the finger bed in an operating element of the vehicle, a reduction of interfering influences of other light sources from outside the opto-electronic sensor arrangements with respect to the measurement or detection. These other light sources, independent of whether these are a direct light irradiation or reflected light, are also referred to as extraneous light.

Embodiments in which the finger bed is integrated in the gear lever knob as an operating element additionally enable an optimum distribution of the contact pressure of the fingertip. By this finger bed, the fingertip is placed, i.e. guided, as optimal as possible on the optical sensor. The gear lever knob clearly offers more room as compared to conventional steering-wheels or steering-wheel rims to integrate the finger bed and the optical sensor (optoelectronic sensor arrangement) and possibly also the electronic circuit for controlling and evaluating the data. Apart from this, the gear lever knob is not as security-relevant as the steering-wheel. This may lead to a higher acceptance from the industry with respect to future technological manufacturing processes.

With embodiments having a finger bed integrated into the gear lever knob, apart from this, complicated or extreme light conditions in the automobile are eliminated or reduced on the one hand by the somewhat lower position of the gear lever knob (compared to the steering-wheel) relative to the windscreen and side windows. Further, the light-sensitive element or photo element of the optoelectronic sensor arrangement in the gear lever knob is protected from extraneous light by the special setup of the finger bed, which encloses the finger flush.

### BRIEF DESCRIPTION OF THE DRAWINGS

In the following, embodiments of the present invention are explained in more detail with reference to the accompanying drawings.

FIG. 1 shows one embodiment of a device for detecting at least one vital parameter of a person in a vehicle in the form of a light remission sensor integrated into a gear lever knob.

FIG. 2 shows the embodiment according to FIG. 1 with the hand of the person applied for whom the at least one vital parameter is to be detected.

FIG. 3 shows an embodiment of an optoelectronic sensor arrangement (here of a light remission sensor) arranged in a finger bed of an operating element.

FIG. 4 shows a device for monitoring at least one vital parameter of a person in a vehicle and a data transmission to a third person, e.g. doctors' pc or server triggered by a threshold value exceedance.

FIGS. 5 and 6 show two photos of an embodiment of a device for detecting at least one vital parameter of a person in the form of a light remission sensor integrated in a gear lever knob.

FIG. 7 shows a photo of a further embodiment of a device for detecting at least one vital parameter of a person in the form of a light remission sensor integrated into a gear lever knob.

FIG. 8 shows an embodiment of an operating surface for medical application.

Here, in the present invention for objects and functional units comprising same or similar functional characteristics, the same reference numerals are used.

### DETAILED DESCRIPTION OF THE INVENTION

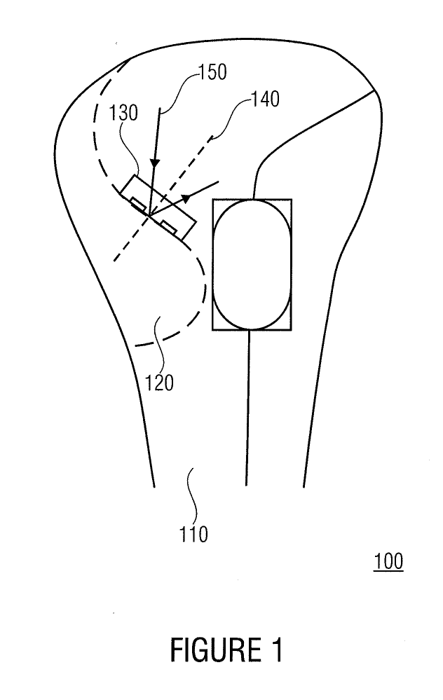

FIG. 1 shows an embodiment of a device 100 for detecting at least one vital parameter of a person in a vehicle.

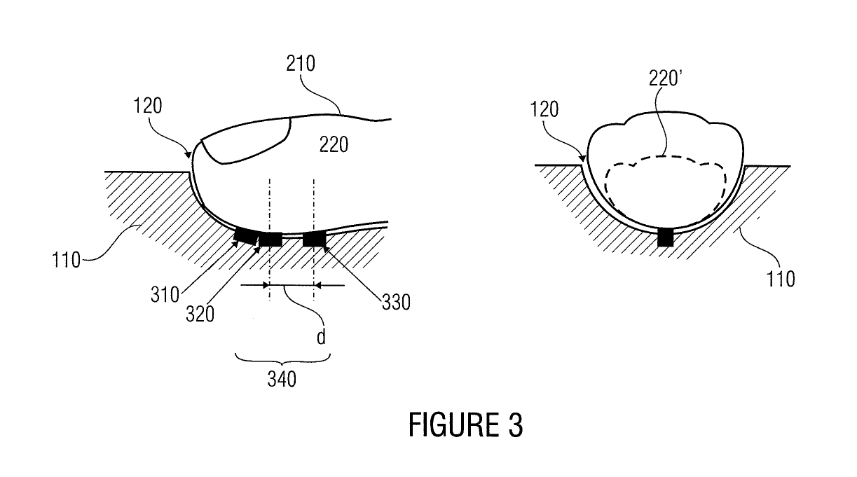

The device 100 for detecting comprises one gear lever knob 110 or knob 110 of a gear lever, comprising a finger bed 120 (see dashed line), wherein in the finger bed 120 a light remission sensor 130 or generally an optoelectronic sensor arrangement 130 is arranged. The light remission sensor 130 integrated in the gear lever knob comprises two light sources, a red diode and an infrared diode, and a photo sensor or a photo diode, as it is explained in the following with reference to FIG. 3. The light sources and the photo sensor lie in the same plane and are located close to each other. The light sources radiate into the tissue of the finger and the photo sensor measures the reflected remitted portions of the light field, as it is schematically illustrated with reference to the perpendicular 140 (dashed line perpendicular to the plane of the sensor area or the area of the finger bed 120 in which the light sources and the photo sensor are arranged) and the indicated radiation path 150 of the reflected portion of the light field.

The sensor 130 is integrated into the finger bed 120, wherein the finger bed 120 is a special recess or trough in the gear lever knob 110 or more general in an operating element of the vehicle into which a finger of the driver may comfortably be inserted. By this finger bed 120, the fingertip is placed as optimally as possible onto the optical sensor 130. Simultaneously, the photo element of a sensor 130 is protected from exterior lighting and the applied force at the contact location between finger and sensor 130 is evenly distributed. One embodiment of the finger bed 120 may here fulfill one or several of the following ergonomic requirements:
comfortable position of the finger,
optimally distributed contact pressure when applying the finger, e.g. by guiding the finger by means of the finger bed,
transverse position of the finger at the gear lever knob possible to consider different gripping habits of the drivers,
dimensions of the finger bed 120 such that independent of the finger size varying from person to person a flush reception of the finger in the finger bed 120 is achieved and interfering influences by extraneous light may be prevented or at least reduced,
(automatic) adaptation of the width of the finger 120 to the finger, and
guaranteeing that the finger may not be clamped in the finger bed, for example by the finger bed being open or at least being partially open at the side opposite to the optoelectronic sensor arrangement and not being tapered towards the top.

An adaptation of the width of the finger bed to the finger may, for example, be achieved by the gear lever knob 110 being exchangeable and in that for different drivers specific gear lever knobs 110 may be mounted with a finger bed 120 adapted to the size of the hand, with a finger bed 120 adapted to the finger and/or the gripping habits of the driver. Here, for example, a corresponding locking guarantees that the interchangeable lever knob 110 is firmly connected to the gear lever. An automatic adaptation of a common gear lever knob 110 for several drivers and thus an automatic adaptation of the width of the finger bed to the different fingers may, for example, be achieved by the use of elastic materials in the area of the finger bed 120, wherein the respective driver-specific setting of the finger bed width is stored and controlled, for example, in a driver profile apart from other driver-specific settings, like the setting of the interior and exterior mirror.

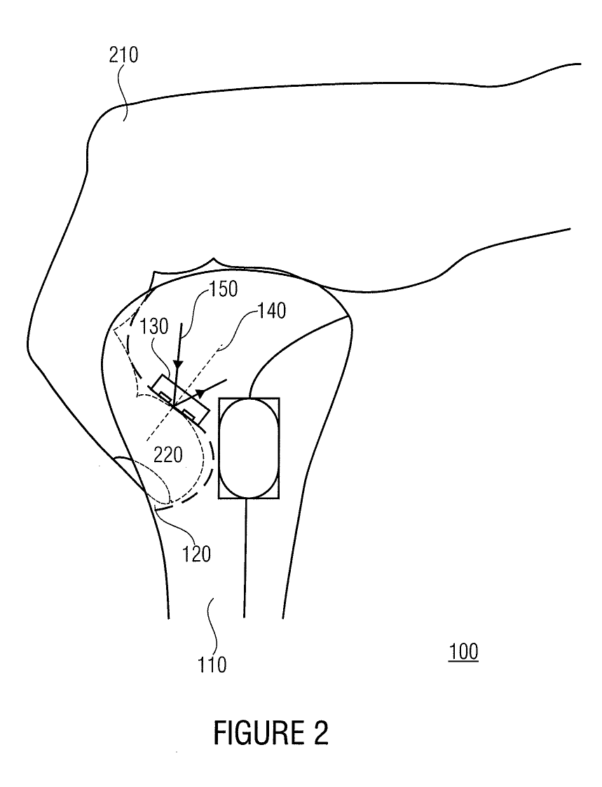

FIG. 2 shows the embodiment of the device 100 for detecting at least one vital parameter according to FIG. 1 with the hand 210 applied and a fingertip 220 applied to the optoelectronic sensor arrangement 130.

FIG. 3 shows an embodiment of a finger bed of an operating element of a vehicle with an optoelectronic sensor arrangement 130 in the form of a light remission sensor integrated into the finger bed 120. The optoelectronic sensor arrangement 130 comprises a red diode 310, an infrared diode 320 and a photodiode 330, which are arranged in a sensor area 340 of the finger bed 120. The distance d of the infrared diode 320 and the photodiode 330 is, for example, 8 mm. As indicated on the right in FIG. 3, the red diode, the infrared diode and the photodiode are arranged in one line. In alternative embodiments, the same may also be arranged differently to each other.

The red diode is, for example, implemented to generate visible light in the 660 nm range and the infrared diode 320 generates light in the wavelength range of 390 nm non visible for humans. Due to the different coloring of the hemoglobin saturated with oxygen, for the irradiating red light or infrared light, a different absorption results which is measured by the photosensor 330, wherein an evaluation unit may, for example, detect or determine the oxygen saturation of the blood in the capillaries by means of a comparison of the measurement results to a reference table. Apart from the oxygen saturation, by means of the optoelectronic sensor arrangement 130, also generally the pulse or the pulse wave, pulse rate, pulse rate variability may be determined. For the determination of these pulse parameters or pulse information, however, in contrast to the determination of the oxygen saturation, one single light source of the two light sources is sufficient.

As explained above, the finger bed 120 is advantageously implemented so that it receives fingers or fingertips of a different size or guides the same, so that the fingertip 220 is applied in the sensor area 340 in order to enable an optimum measurement. This may, for example, be achieved by a concave finger bed 120 as illustrated on the right in FIG. 3. For illustration purposes, a smaller fingertip 220′ (dashed line) is indicated on the right in FIG. 3. A concave finger bed additionally enables a flush fitting of fingers of different sizes in order to reduce interfering influences by extraneous lights.

FIGS. 1-3 in other words generally show a device for detecting at least one vital parameter of a person in a vehicle, comprising: an optoelectronic sensor arrangement 130 for detecting the at least one vital parameter by means of light remission, wherein the optoelectronic sensor arrangement comprises at least one light source 310, possibly a second light source 320 and a light-sensitive element 330, and wherein the first light source 310, the second light source 320 and the light-sensitive element 330 are arranged in a sensor area 340 a finger bed 120 of an operating element 110 of the vehicle, and wherein the finger bed 120 is implemented to integrate a fingertip 220 of the person flush in a sensor area 340 of the finger bed 120 in which the first light source 310, the second light source 320 and the light sensitive element 330 are arranged.

Here, the optoelectronic sensor arrangement 130 according to FIGS. 1-3 comprises a light remission sensor, wherein the red diode 310 corresponds to the first light source, the infrared diode 320 corresponds to the second light source and the photo sensor or the photodiode 330 corresponds to the light-sensitive element. Generally speaking, the first light source is implemented to generate light of a first wavelength, the second light source is implemented to generate light of a second wavelength, wherein the first and the second wavelengths are different to each other.

Further, embodiments of the finger bed may be described as a recess open to one side in the operating element, which is implemented to receive a bottom side of the fingertip 220 flush. The opening on one side of the finger bed 120 has the advantage as compared to clips or arrangements completely enclosing the finger bed that, for example, in traffic the finger may, at any time, be removed from the finger bed without getting stuck. Further, the first light source 310, the second light source 320 and the light-sensitive element 330 are arranged on the same side of the finger with respect to the finger, and the light-sensitive element 330 is implemented to receive the reflected, remitting light of the first and second light sources 310, 320.

Although FIGS. 1 and 2 show an embodiment of the device 100 for detecting at least one vital parameter, wherein the operating element which is integrated into the finger bed 120 is a gear lever knob, in alternative embodiments the operating element may, for example, also be the steering-wheel or the finger bed may be integrated into the steering-wheel or into the middle arm rest or the finger bed may be integrated in an area of the middle arm rest.

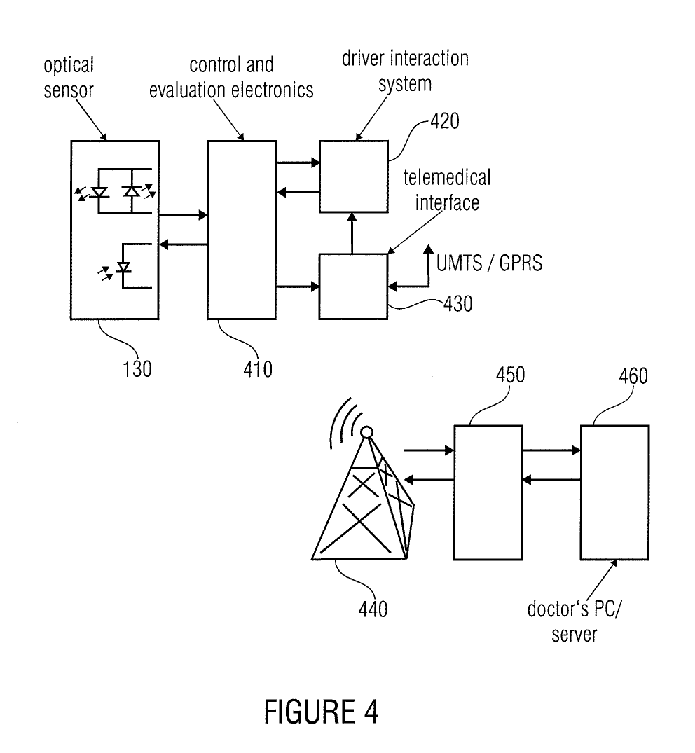

FIG. 4 shows an embodiment of a measurement system for the detection of pulse waves, pulse rate, pulse rate variability and oxygen saturation of blood of a person in a vehicle. The measurement system consists of an optical sensor 130, a control and evaluation electronics 410, a driver interaction system 420, a tele-medical interface 430 and a doctor's PC (personal computer) 460. Here, the optical sensor 130, as explained above with reference to FIGS. 1-3, is arranged in a finger bed in an operating element of the vehicle and the control and evaluation electronics 410, the driver interaction system 420 and the tele-medical interface 430 are also integrated into the vehicle.

In a further embodiment, for example, also the control and evaluation electronics 410 may be integrated in the same operating element as the optical sensor 130. The doctor's PC or the corresponding server is, for example, setup at a doctor's, a hospital or another place, and connected, for example, by means of a mobile radio network 450, also referred to as a mobile core network, and a base station 440 with a mobile transmit/receive unit integrated in the tele-medical interface 330. The mobile radio network may, for example, be a UMTS mobile radio network (Universal Mobile Telecommunication Standard) or also be based on another mobile radio standard like e.g. GPRS (Global Packet Radio System) or GSM (Global System for Mobile Communication) or another radio communications network, e.g. WLAN (Wireless Local Area Network).

The detected vital parameters are classified by the evaluation electronics 410, for example, into three warning groups (see table). For each of the warning groups, the top and bottom threshold value of the corresponding vital parameters are defined. Also the warning stages are defined by means of the grade of danger for the driver for each warning group. When exceeding the given threshold value of the vital parameters, the corresponding warning message is triggered.

TABLE
Definition of the warning stages.

| | |
|---|---|
| N/Data | The first warning stage corresponds to the normal state |
| Errors | of the driver (the vital parameter is within normal |
| | physiological limits). |
| | The non-evaluable or lost sensor data is also encoded as a first |
| | warning stage. |
| Attention | The second warning stage corresponds to the driver state when |
| | attention has to be called to the values of the vital parameters. |
| | The warning message regarding the exceeded threshold values |
| | follows. |
| | The driver is requested to leave traffic. |
| Critical | The third warning stage corresponds to the driver's state when |
| | medical assistance is needed. |
| | The warning message regarding the exceeded threshold values |
| | follows. |
| | The driver has to leave traffic. |

In other words, a further embodiment of the present invention provides a device for monitoring at least one vital parameter of a person in a vehicle, comprising: a device 100 for detecting the at least one vital parameter as it was described with reference to FIGS. 1-3; and at least one device 410 for evaluating the at least one vital parameter, which is implemented to compare one value of the at least one vital parameter to at least one given threshold value and to output a warning message when the value of the at least one vital parameter exceeds or falls below this at least one threshold value.

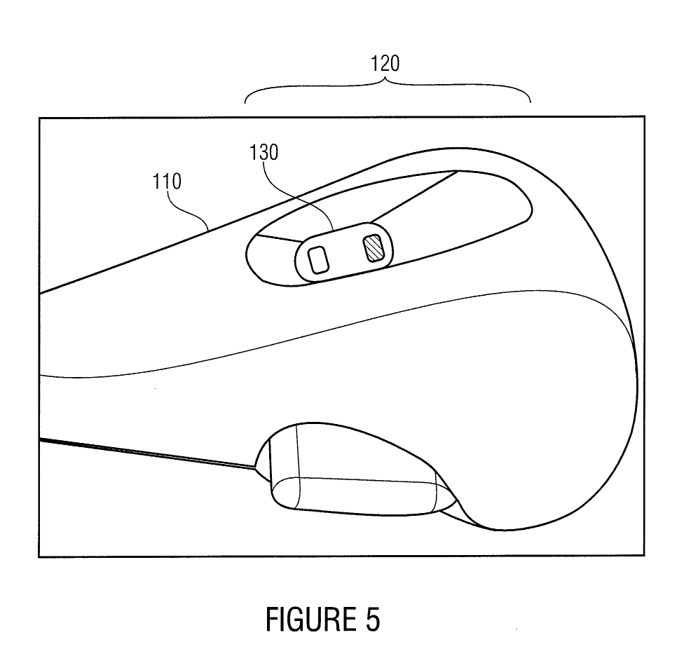

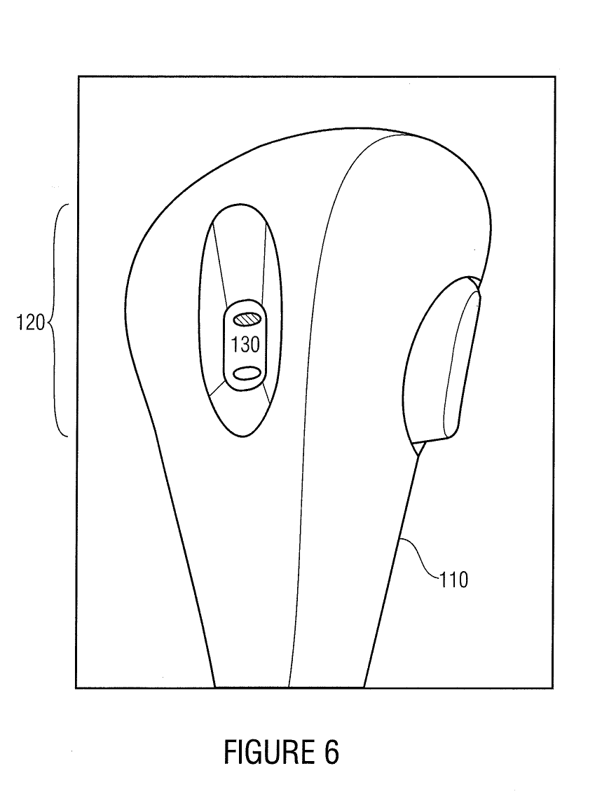

FIGS. 5 and 6 show photos of an exemplary integration of an optical sensor 130 into a finger bed 120 which is again integrated into a gear lever knob 110.

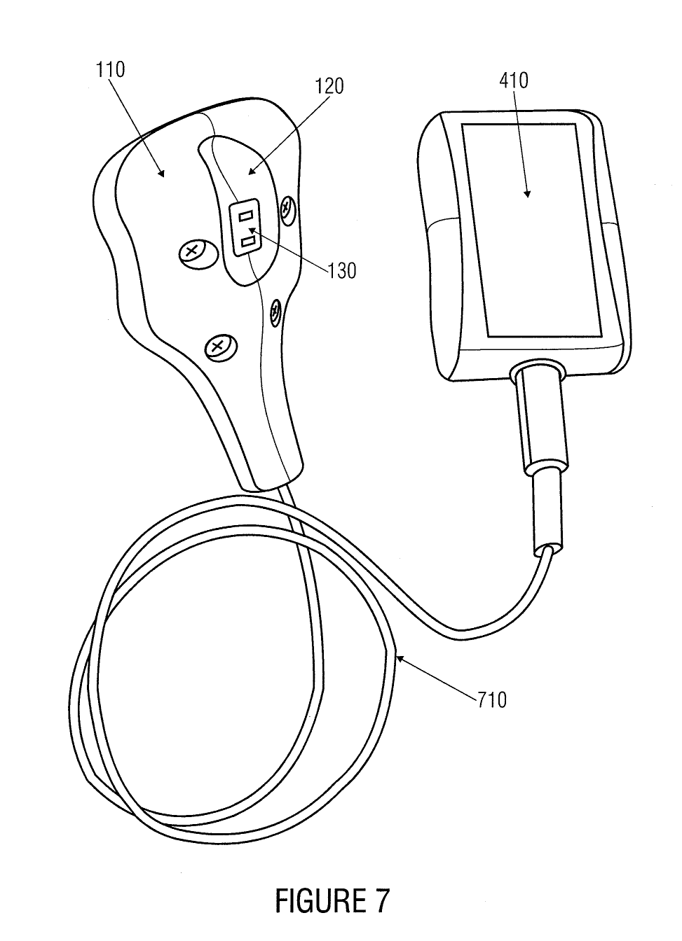

FIG. 7 shows a photo of a prototype of an optical sensor 130 integrated into the finger bed 120 of a gear lever knob 110 which is connected to a control and evaluation electronics 410 by means of a multi-core shielded cable. The control and evaluation electronics 410 is coupled to a control unit, for example, the driver interaction system 420 and the tele-medical interface 430 (see FIG. 4) via a Blutooth interface integrated in the electronic circuit of the control and evaluation electronic 410.

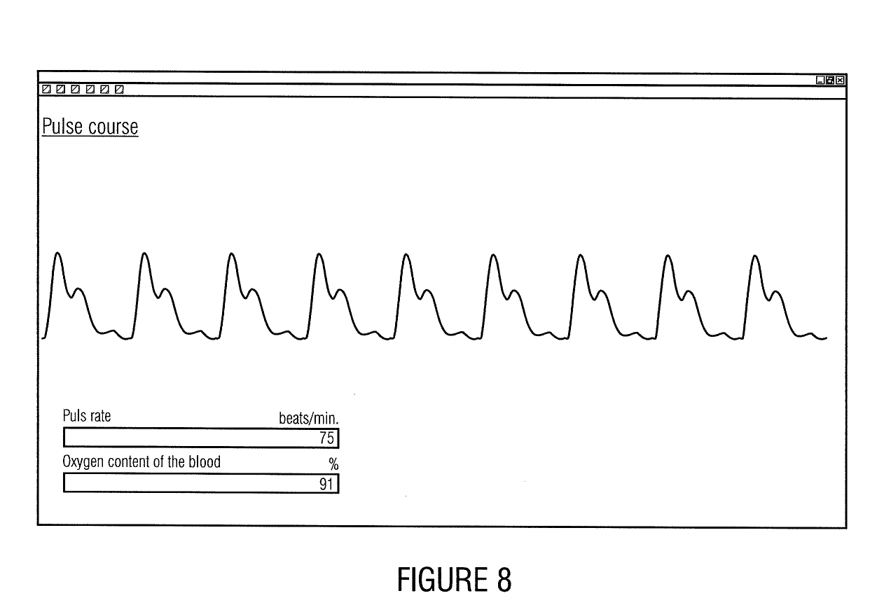

FIG. 8 shows an example of an operating surface or user interface for a medical treatment as it may, for example, be executed on the doctor's PC 460 which, as illustrated in FIG. 8, may, for example, indicate the pulse course over time and the pulse rate, e.g. 75 beats per minute (see FIG. 8), and the oxygen content of the blood, e.g. 91% (see FIG. 8).

With reference to the previous explanations, it may be noted that embodiments of the present invention realize a “method and measurement system for the optical detection of the pulse wave, pulse rate, pulse rate variability and oxygen saturation of the blood of a driver in the gear lever knob of a vehicle”, a “measurement system for the detection of the pulse wave, pulse rate, pulse rate variability and oxygen saturation of the blood of a driver of a vehicle by means of an optical sensor integrated in the gear lever knob” and/or an “optical sensor for the detection of the pulse wave, pulse rate, pulse rate variability and oxygen saturation of the blood of a driver in the gear lever knob”. Here, embodiments of the present invention may be referred to both as a medical system for monitoring the vital parameters of a person, in particular, the detection of the pulse wave, the pulse rate, the pulse rate variability and the oxygen saturation of the blood and also as a means and a method for passenger protection or passenger warning and driver support. Here, the field of application of the invention, for example, is in the area of preventive, monitoring and supporting medicine for the use in the vehicle, key word “driver assistance system”, under or without telemetric medical monitoring. Here, embodiments of the present invention may be used to monitor drivers with cardiologic risk factors when driving with respect to fatigue, attention, stress in everyday life.

Here, embodiments of the present invention further provide a method and a measurement system enabling executing the measurements of the pulse wave parameters, the pulse rate, the pulse rate variability and the oxygen saturation of the blood under automobile conditions with a restricted impairment of the driver. Here, the measurement values are made accessible for the driver, for example, via a driver interaction system and for example for an attending physician via a telemetric interface. The detected values may be classified and be matched with a personal driver profile. When exceeding the threshold values, for example, a warning message or an alarm or an emergency service call is triggered.

Embodiments of the present invention further relate to a device of an optical sensor in the gear lever knob of a vehicle for the detection of the pulse wave with different electromagnetic wavelengths in the field of the non-visible infrared and the visible red electromagnetic spectrum.

Further, embodiments of the present invention provide a device for monitoring a pulse rate, pulse rate variability and oxygen saturation of the blood of the driver in the automobile based on the detected pulse wave and/or a measurement system for the detection of the pulse wave, pulse rate, pulse rate variability and the oxygen saturation of the blood of the driver in the automobile.

Further, embodiments of the present invention provide a driver assistance system for the medical monitoring of the health of the driver, in particular, the pulse wave profile, the pulse rate, the pulse rate variability and oxygen saturation of the blood. Here, further fields of application result, e.g. by coupling of influences on the driver, like e.g. stress, to a corresponding assistance performance, for example a corresponding request by means of an announcement or indication to the driver to reduce speed.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

1. A device for detecting at least one vital parameter of a person in a vehicle, comprising:
an optoelectronic sensor arrangement for detecting the at least one vital parameter by means of light remission, wherein the optoelectronic sensor arrangement comprises at least one light source and a light-sensitive element, wherein the first light source and the light-sensitive element are arranged in a finger bed of an operating element of the vehicle, wherein the finger bed is implemented to integrate a fingertip of the person flush in a sensor area of the finger bed in which the first light source and the light sensitive element are arranged, and wherein the finger bed is a recess open to one side in the operating element, which is implemented to receive a bottom side of the fingertip flush.

2. The device according to claim 1, wherein the first light source and the light-sensitive element are arranged in the same plane of the finger bed.

3. The device according to claim 1, wherein the operating element is a knob of a gear lever.

4. The device according to claim 1, wherein the vital parameter is a pulse information.

5. The device according to claim 1, wherein the optoelectronic sensor arrangement additionally comprises a second light source arranged within the finger bed of the operating element.

6. The device according to claim 5, wherein the vital parameter is an oxygen saturation of the blood of the person.

7. A device for monitoring at least one vital parameter of a person in a vehicle, comprising:
a device for detecting the at least one vital parameter of a person in a vehicle, comprising:
an optoelectronic sensor arrangement for detecting the at least one vital parameter by means of light remission, wherein the optoelectronic sensor arrangement comprises at least one light source and a light-sensitive element, wherein the first light source and the light-sensitive element are arranged in a finger bed of an operating element of the vehicle, wherein the finger bed is implemented to integrate a fingertip of the person flush in a sensor area of the finger bed in which the first light source and the light sensitive element are arranged, and wherein the finger bed is a recess open to one side in the operating element, which is implemented to receive a bottom side of the fingertip flush; and
a device for evaluating the at least one vital parameter which is implemented to compare a value of the at least one vital parameter to at least one given threshold value and to output a warning message when the value of the at least one vital parameter exceeds or falls below this at least one threshold value.

8. The device according to claim 7, comprising:
a driver assistance system;
wherein the device for evaluating is implemented, by means of exceeding or falling below the at least one threshold value, to detect cardiologic risk factors or a chronic lung disease of a person, detect fatigue and/or stress of the person, and the driver assistance system is implemented, when exceeding or falling below the at least one threshold value of the person, to offer or execute an assistance service depending thereon.
